(19) United States
(12) Patent Application Publication
(10) Pub. No.: US 2018/0273454 A1
(43) Pub. Date: Sep. 27, 2018

(54) MULTICOMPONENT HETEROGENEOUS CATALYSTS FOR DIRECT CO2 HYDROGENATION TO METHANOL

(71) Applicant: SABIC GLOBAL TECHNOLOGIES B.V., BERGEN OP ZOOM (NL)

(72) Inventors: Khalid A. ALMUSAITEER, Thuwal (SA); Ahmed AL-HADHRAMI, Thuwal (SA); Abdulaziz KHAN, Thuwal (SA); Abdulaziz AL-AMER, Thuwal (SA)

(21) Appl. No.: 15/761,385

(22) PCT Filed: Sep. 13, 2016
(86) PCT No.: PCT/IB2016/055453

§ 371 Date: Mar. 19, 2018

Related U.S. Application Data
(60) Provisional application No. 62/232,039, filed on Sep. 24, 2015.

(51) Int. Cl. C07C 29/154; B01J 23/83; B01J 23/00; B01J 37/04; B01J 37/03; B01J 37/08; B01J 37/18

(52) U.S. Cl. CPC C07C 29/154; B01J 23/83; B01J 23/002; B01J 2523/00; B01J 37/031; B01J 37/08; B01J 37/18; B01J 37/04

(57) ABSTRACT

Mixed metal oxide catalysts capable of catalyzing hydrogenation of carbon dioxide to methanol reaction are disclosed, as well as a method for producing methanol from carbon dioxide and hydrogen. The mixed metal oxide catalysts include copper (Cu), and M¹ and M² oxides. M¹ can be zinc (Zn), zirconium (Zr), or cerium (Ce), or any combination thereof, and M² can be yttrium (Y), barium (Ba), rubidium (Rb), terbium (Tb), strontium (Sr), or molybdenum (Mo), or any combination thereof, with the proviso that M² is not Y when the mixed metal oxide catalyst is [Cu/Zn/M²]0ₙ or [Cu/Zr/M]0ₙ, where n is determined by the oxidation states of the other elements.

### CROSS REFERENCE TO RELATED APPLICATIONS

This application claims the benefit of priority of U.S. Provisional Patent Application No. 62/232,039, filed Sep. 24, 2015, which is hereby incorporated by reference in its entirety.

### BACKGROUND OF THE INVENTION

A. Field of the Invention

The invention generally concerns catalysts capable of catalyzing a hydrogenation reaction that directly hydrogenates carbon dioxide to produce methanol. In particular, a multicomponent heterogeneous catalyst composition containing mixed metal oxides is used to catalyze the production of methanol from carbon dioxide. The catalysts have good activity and selectivity. By-products produced from the reaction can be minimized to carbon monoxide and water.

B. Description of Related Art

Carbon dioxide (CO₂) is mostly produced as a waste by-product in oil refinery, fossil fuels combustion, and chemicals production. Many natural gas sources contain sizeable concentrations (as much as 50 vol. %) of CO₂. Most of the CO₂ produced in above processes is released into the atmosphere. However, to mitigate CO₂ emissions and their adverse effects on the global climate, many efforts have been undertaken to develop new technologies and upgrade the current ones that would prevent or reduce CO₂ generation. In addition, capturing the generated CO₂ and using it for various applications such as in an enhanced oil recovery process or as an alternative feedstock and building block for several important chemicals has been investigated as an outlet for waste CO₂.

One method to use carbon dioxide is to produce methanol. As shown in reaction scheme (1), carbon dioxide can be hydrogenated using copper catalysts to produce methanol.

CO₂+3H₂↔CH₃OH+H₂O ΔH=−49.43 kJ/mol (1)

In this process, methanol formation is favored by lower temperature (less than 250° C.) and higher pressure using copper catalysts. At higher temperatures formation of other by-products besides methanol occurs, thus decreasing the amount of methanol formed. Further, deactivation of the copper catalysts can occur through formation of water on the active copper sites. Commercially, this problem has been addressed using a two-step process referred to as the CAMERE process (carbon dioxide hydrogenation to form methanol via a reverse-water gas shift reaction or RWGSR). In the CAMERE process, two reactors are consecutively arranged to convert carbon dioxide to CO and H₂O in the first reactor by RWGSR (reaction scheme (2)). Water and, optionally carbon dioxide are then removed to form a stream rich in carbon monoxide. The enriched carbon dioxide stream is then fed into the second reactor to produce methanol under catalytic conditions (See, reaction scheme (3)).

CO₂+2H₂→CO+2H₂O (2)

CO+2H₂→CH₃OH (3)

In this approach, RWGSR can be carried out at high temperature (>600° C.) under catalytic conditions to obtain high CO₂ conversion to CO. Conversion of CO to methanol in a second reactor can lead to high methanol productivity due to the removal of water. Other approaches to hydrogenate CO₂ include varying catalyst compositions and varying methods of preparation. By way of example, U.S. Pat. No. 5,393,793 by Inui discloses the use of CuO/ZnO/Cr₂O₃/Al₂O₃/La₂O₃ to of afford methanol in a 16.2% yield at a H₂/CO₂ equal to 3, at a temperature of 250° C., a pressure of 50 bar, and space velocity of 4700 h⁻¹. In another example, Energy Conversion and Management (1992), 33, pp. 521-528 by Arakawa describes using a 43% copper-20% zinc oxide-34% alumina and 3% chromium oxide catalyst at a H₂/CO₂ of 3 at a temperature of 250° C., a pressure of 70 bar, and space velocity of 1800 h⁻¹ that results in an increase in methanol yield to 19.9%. Chinese Patent Publication CN102240553 by Zhou et al. describes a catalyst for synthesizing methanol by hydrogenating carbon dioxide with a mixed oxide of one or more of Cu, Zn, La, Ce and M, wherein M is Al, Si, Ti or Zr. The disclosed catalyst includes the following components in percentage by weight: 30-70% of CuO, 10-40% of ZnO, 1-5% of Ln₂O₃, 1-5% CeO₂ and 5-20% of carrier oxides MxOy. Still other approaches to increase methanol production include changing the type or combination of active components, supports, promoters, preparation methods, and surface morphology (See, for example, Gao et al. in American Chemical Society, Division of Fuel Chemistry (2012), 57(1), pp. 280-281 and Toyir et al. in Applied Catalysis B (2001), 29, pp. 207-215 and Applied Catalysis B (2001), 34, pp. 255-266).

Most of the above-mentioned processes suffer from poor selectivity, increased formation of by-products, and decreased methanol yields or combinations thereof.

### SUMMARY OF THE INVENTION

A discovery has been made that solves the problems associated with the production of methanol from carbon dioxide. Notably, the discovery uses a copper containing catalyst capable of catalyzing the direct hydrogenation of carbon dioxide (CO₂) to (MeOH) reaction in one pass, which eliminates the need for a multi-step reaction process. In particular, the catalysts can be nanosized catalysts that have water inhibition properties, which eliminate the need to remove water from the process of hydrogenating carbon dioxide to methanol. The catalysts of the present invention have also shown increased selectivity and activity towards the production of methanol from carbon dioxide as well as increased catalyst stability during prolonged periods of use. By way of example, the catalysts of the present invention can be multicomponent catalysts that include various Cu loadings, M¹ oxides, and M² oxides. M¹ oxide can include zinc (Zn), zirconium (Zr), or cerium (Ce), or combinations thereof. M² oxides can include yttrium (Y), barium (Ba), rubidium (Rb), terbium (Tb), strontium (Sr), or molybdenum (Mo), or combinations thereof. Notably, it was found that the presence of M¹ in the catalysts of the present invention can promote oxygen storage and release and also reduce or inhibit water from depositing on the active catalytic sites. Still further, it is believed that the presence of Y, La, Ba, or Rb oxides increase the methanol yield due to a strong interaction with the CO and O atoms of CO₂ (due to large surface reconstruction). It was also discovered that preparation of the catalysts using a gel oxalate co-precipitation method provided nanoparticles of a desired shape and size, thus allowing for a higher interaction with CO₂, resulting in a higher conversion of CO₂ to methanol.

In one aspect of the present invention, there is disclosed a mixed metal oxide catalyst capable of directly producing methanol from carbon dioxide and hydrogen (H₂). The mixed metal oxide catalyst can include copper (Cu), M¹ oxides, and M² oxides, where M¹ can be Zn, Zr, Ce, or any combination thereof, and M² is Y, Ba, Rb, Tb, Sr, or Mo, or any combination thereof, with the proviso that M² is not Y when the mixed metal oxide catalyst is [Cu/Zn/M²]Oₙ or [Cu/Zr/M²]Oₙ, where n is determined by the oxidation states of the other elements. The catalyst can be [Cu/Zn/Zr/Ce/M²]Oₙ mixed metal oxide having a general formula of: [CuₐZn_{b}Zr_{c}Ce_{d}Mₑ²]Oₙ where a is 25 to 80, b is 1 to 57, c is 1 to 30, d is 1 to 30, and e is 1 to 40. M² of the mixed metal oxide catalyst can be Y, Ba, Rb, Tb, Sr, Mo or a mixture thereof. In some aspects the catalyst is a [Cu/Zn/M²]Oₙ mixed metal oxide having a general formula of [CuₐZn_{b}M_{c}²]Oₙ, where a is 25 to 80, b is 1 to 57, and c is 1 to 30. In another aspect of the invention, the mixed metal catalyst can be a [CuₐZr_{b}M_{c}²]Oₙ metal oxide having the general formula of [CuₐZr_{b}M_{c}²]Oₙ where a is 25 to 80, b is 1 to 57, and c is 1 to 30. The catalyst of the present invention can be a bulk metal oxide catalyst. The catalyst can be the reaction product of co-precipitation of metals, or salts thereof, from a solution containing oxalic acid followed by calcination of the precipitate. In some aspects, the catalyst has been calcined for 2 to 6 hours at a temperature of 250 to 650° C. In some aspects of the invention, the calcined catalyst can be reduced for 2 to 3 hours at a temperature of 180 to 350° C. In a particular aspect, the catalyst is capable of producing CH₃OH from CO₂ and H₂ in a single pass such that CO₂ is directly hydrogenated to CH₃OH. The by-products produced from the reaction can be limited to carbon monoxide and water.

Also disclosed are methods of producing methanol from carbon dioxide and hydrogen using the mixed metal oxide catalyst of the present invention. Such methods include contacting a reactant gas stream that includes CO₂ and H₂ with the mixed metal oxide catalyst of the present invention under conditions sufficient to produce a product gas stream that includes CH₃OH. The method permits the production of CH₃OH from the CO₂ and H₂ in a single pass via direct hydrogenation of CO₂. The presence of by-products in the CH₃OH product gas stream can be limited to carbon monoxide and water. The methanol and water produced in the reaction can be condensed and separated. In some aspects, the reactant gas stream has a hydrogen to carbon dioxide ratio (H₂/CO₂) of 1 to 5, preferably 3 to 5 and the reaction conditions include a temperature of 200° C. to 300° C., preferably, 220° C. to 260° C., a pressure of 1 bar to 100 bar, preferably 30 bar to 50 bar, and a gas hourly space velocity of 2,500 h⁻¹ to 20,000 h⁻¹, preferably of 4,000 h⁻¹ to 6,000 h⁻¹. The single pass methanol selectivity can be 10 to 100%, preferably, 30 to 90%, or more preferably from 50 to 85% after 100 hours to 800 hours on the stream. The single pass CO₂ conversion can be 5% to 60% after 110 hours to 800 hours on the stream. In one aspect of the methods, the catalyst remains 90 to 99% active, preferably 94 to 98% active, after 350 hours of time on the stream.

Also disclosed is a system for producing methanol from carbon dioxide and hydrogen. The system can includes an inlet for a reactant feed that includes CO₂ and H₂; a reaction zone that is configured to be in fluid communication and/or in contact with the inlet, and an outlet configured to be in fluid communication and/or in contact with the reaction zone and configured to remove a first product stream including CH₃OH from the reaction zone. The reaction zone includes the mixed metal oxide catalyst. The reaction zone of the system can also include the reactant feed and the first product stream. In some aspects, the reaction zone can be a continuous flow reactor, a fixed-bed reactor, a fluidized reactor, or a moving bed reactor.

In another aspect, a method of making a mixed metal oxide catalyst is provided. The method can include obtaining a first solution containing metal precursor materials, obtaining a second solution containing oxalic acid dissolved in an alcohol, mixing the first and second solution together to form a precipitate from the metal precursor materials, and calcining the precipitate to obtain the mixed metal oxide catalyst of the present invention. The metal precursor materials can include Cu, M¹ (e.g., Zn, Zr, Ce, or any combination thereof), and M² (e.g., Y, Ba, Rb, Tb, Sr, Mo, or any combination thereof). In a particular aspect, the metal precursor materials can be nitrate salts of Cu, M¹, and M² and the formed precipitate can be calcined (e.g., for 2 to 6 hours at a temperature of 250 to 650° C.). The calcined mixed metal oxide catalyst can be reduced (e.g., for 2 to 3 hours at a temperature of 180 to 350° C.) prior to use.

In the context of the present invention, 35 embodiments are described. Embodiment 1 describes a mixed metal oxide catalyst capable of producing methanol (CH₃OH) from carbon dioxide (CO₂) and hydrogen (H₂), the mixed metal oxide catalyst comprising copper (Cu), M¹, and M², wherein: M¹ is zinc (Zn), zirconium (Zr), or cerium (Ce), or any combination thereof, and M² is ytterbium (Y), barium (Ba), rubidium (Rb), terbium (Tb), strontium (Sr), or molybdenum (Mo), or any combination thereof, with the proviso that M² is not Y when the mixed metal oxide catalyst is [Cu/Zn/M²]Oₙ or [Cu/Zr/M²]Oₙ, where n is determined by the oxidation states of the other elements. Embodiment 2 is the mixed metal oxide catalyst of embodiment 1, wherein the catalyst is a [Cu/Zn/Zr/Ce/M²]Oₙ mixed metal oxide. Embodiment 3 is the mixed metal oxide catalyst of embodiment 2, having a general formula of: [CuₐZn_{b}Zr_{c}Ce_{d}Mₑ²]Oₙ where a is 25 to 80, b is 1 to 57, c is 1 to 30, d is 1 to 30, and e is 1 to 40. Embodiment 4 is the mixed metal oxide catalyst of embodiment 3, wherein M² is Y. Embodiment 5 is the mixed metal oxide catalyst of embodiment 3, wherein M² is Ba. Embodiment 6 is the mixed metal oxide catalyst of embodiment 3, wherein M² is Rb. Embodiment 7 is the mixed metal oxide catalyst of embodiment 3, wherein M² is Tb. Embodiment 8 is the mixed metal oxide catalyst of embodiment 3, wherein M² is Sr. Embodiment 9 is the mixed metal oxide catalyst of embodiment 3, wherein M² is Mo. Embodiment 10 is the mixed metal oxide catalyst of embodiment 1, wherein the catalyst is a [Cu/Zn/M²]Oₙ mixed metal oxide. Embodiment 11 is the mixed metal oxide catalyst of embodiment 10, having a general formula of: [CuₐZn_{b}M_{c}²]Oₙ where a is 25 to 80, b is 1 to 57, and c is 1 to 30. Embodiment 12 is the mixed metal oxide catalyst of embodiment 1, wherein the catalyst is a [Cu/Zr/M²]Oₙ mixed metal oxide. Embodiment 13 is the mixed metal oxide catalyst of embodiment 12, having a general formula of: [CuₐZr_{b}M_{c}²]Oₙ where a is 25 to 80, b is 1 to 57, and c is 1 to 30. Embodiment 14 is the mixed metal oxide catalyst of any one of embodiments 1 to 13, wherein the catalyst has been calcined for 2 to 6 hours at a temperature of 250 to 650° C. Embodiment 15 is the mixed metal oxide catalyst of embodiment 14, wherein the catalyst has been reduced for 2 to 3 hours at a temperature of 180 to 350° C. Embodiment 16 is the mixed metal oxide catalyst of any one of embodiments 1 to 15, wherein the catalyst is a bulk oxide catalyst. Embodiment 17 is the mixed metal oxide catalyst of any one of embodiments 1 to 16, wherein the catalyst is the reaction product of co-precipitation of metals, or salts thereof, from a solution containing oxalic acid followed by calcination of the precipitate. Embodiment 18 is the mixed metal oxide catalyst of any one of embodiments 1 to 17, wherein the catalyst is capable of producing CH₃OH from CO₂ and H₂ in a single pass.

Embodiment 19 is a method of producing methanol (CH₃OH) from carbon dioxide (CO₂) and hydrogen (H₂), the method comprising contacting a reactant gas stream that includes CO₂ and H₂ with a mixed metal oxide catalyst of any one of embodiments 1 to 18 under conditions sufficient to produce a product gas stream comprising CH₃OH from hydrogenation of the CO₂. Embodiment 20 is the method of embodiment 19, wherein CH₃OH is produced from the CO₂ and H₂ in a single pass. Embodiment 21 is the method of any one of embodiments 19 to 20, wherein the product gas stream includes by-products consisting of carbon monoxide (CO) and water. Embodiment 22 is the method of any one of embodiments 19 to 21, wherein the methanol and water product are condensed and separated. Embodiment 23 is the method of any one of embodiments 19 to 22, wherein the reactant gas stream has a ratio of H₂/CO₂ of 1 to 5, preferably 3 to 5. Embodiment 24 is the method of any one of embodiments 19 to 23, wherein the reaction conditions include a temperature of 200° C. to 300° C., preferably, 220° C. to 260° C., a pressure of 1 bar to 100 bar, preferably 30 bar to 50 bar, and a gas hourly space velocity of 2,500 h⁻¹ to 20,000 h⁻¹, preferably of 4,000 h⁻¹ to 6,000 h⁻¹. Embodiment 25 is the method of embodiment 24, wherein the single pass CH₃OH selectivity is 10 to 100%, preferably, 30 to 90%, or more preferably from 50 to 85% after 100 hours to 800 hours on the stream. Embodiment 26 is the method of any one of embodiments 24 to 25, wherein the single pass CO₂ conversion is 5% to 60% after 110 hours to 800 hours on the stream. Embodiment 27 is the method of any one of embodiments 19 to 26, wherein the catalyst remains 90 to 99% active, preferably 94 to 98% active, after 350 hours of time on the stream.

Embodiment 28 is a system for producing methanol (CH₃OH) from carbon dioxide (CO₂) and hydrogen (H₂), the system can include: an inlet for a reactant feed comprising CO₂ and H₂; a reaction zone that is configured to be in fluid communication with the inlet, wherein the reaction zone comprises the mixed metal oxide catalyst of any one of embodiments 1 to 17; and an outlet configured to be in fluid communication with the reaction zone and configured to remove a first product stream comprising CH₃OH from the reaction zone. Embodiment 29 is the system of embodiment 28, wherein the reaction zone further comprises the reactant feed and the first product stream. Embodiment 30 is the system of any one of embodiments 28 to 29, wherein the reaction zone is in contact with the inlet, the outlet is contact with the reaction zone, or a combination thereof. Embodiment 31 is the system of any one of embodiments 28 to 30, wherein the reaction zone is a continuous flow reactor selected from a fixed-bed reactor, a fluidized reactor, or a moving bed reactor.

Embodiment 32 is a method of making a mixed metal oxide catalyst of any one of embodiments 1 to 18, the method can include (a) obtaining a first solution comprising metal precursor materials that include copper (Cu), M¹, and M², wherein M¹ is zinc (Zn), zirconium (Zr), or cerium (Ce), or any combination thereof, and M² is ytterbium (Y), barium (Ba), rubidium (Rb), terbium (Tb), strontium (Sr), or molybdenum (Mo), or any combination thereof; (b) obtaining a second solution comprising oxalic acid dissolved in an alcohol; (c) mixing the first and second solution together to form a precipitate from the metal precursor materials; and (d) calcining the precipitate to obtain the mixed metal oxide catalyst of any one of embodiments 1 to 18. Embodiment 33 is the method of embodiment 32, wherein the metal precursor materials are nitrate salts of Cu, M¹, and M². Embodiment 34 is the method of any one of embodiments 32 to 33, wherein precipitate is calcined for 2 to 6 hours at a temperature of 250 to 650° C. Embodiment 35 is the method of embodiment 34, wherein the mixed oxide catalyst is reduced for 2 to 3 hours at a temperature of 180 to 350° C.

The term “mixed metal oxide” catalyst refers to a catalyst that can include metals substantially as oxides or a mixture of metal oxides and metals in other forms (e.g., reduced metal form).

The term “bulk metal oxide catalyst” or “bulk mixed metal oxide catalyst” as that terms are used in the specification and/or claims, means that the catalyst includes metals, and does not require a carrier or a support.

The term “conversion” means the mole fraction (i.e., percent) of a reactant converted to a product or products.

The term “selectivity” refers to the percent of converted reactant that went to a specified product, for example, methanol selectivity is the % of CO₂ that formed methanol.

The term “about” or “approximately” are defined as being close to as understood by one of ordinary skill in the art. In one non-limiting embodiment, the terms are defined to be within 10%, preferably within 5%, more preferably within 1%, and most preferably within 0.5%.

The term “substantially” and its variations are defined to include the ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The terms “wt. %”, “vol. %”, atomic (at.)% or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

The catalysts and methods of the present invention can “comprise,” “have”, “include”, “consist essentially of,” or “consist of” particular ingredients, components, compositions, etc. disclosed throughout the specification. With respect to the transitional phase “consisting essentially of,” in one non-limiting aspect, a basic and novel characteristic of the catalysts of the present invention is their ability to catalyze the direct hydrogenation of carbon dioxide to produce methanol.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIG. 1 is a schematic of an embodiment of a system for producing methanol from CO₂.

FIG. 2 shows X-ray Diffraction (XRD) patterns of fresh and used catalyst of the present invention with an atomic ratio of copper, zinc, zirconium, cerium and yttrium of 60:20:10:5:5.

FIG. 3 shows X-ray Diffraction (XRD) patterns of fresh and used catalyst of the present invention with an atomic ratio of copper, zinc, zirconium, cerium and lanthanum of 65:20:5:5:5.

FIG. 4 shows an X-ray Diffraction (XRD) pattern of the catalyst of the present invention with an atomic ratio of copper, zinc, zirconium, cerium and barium of 55:10:15:10:10.

FIG. 5 shows an X-ray Diffraction (XRD) pattern of the catalyst of the present invention with an atomic ratio of copper, zinc, zirconium, cerium and barium of 60:15:10:10:5.

FIG. 6 shows a graphical representation of the methanol yield over Cu/Zn/Zr/Ce/Y catalysts of the present invention as a function of time on stream (TOS) at 30 bar (3.0 MPa) and different temperature and GHSV.

FIG. 7 shows a graphical representation of the methanol yield over a 60% Cu/20% Zn/10% Zr/5% Ce/5% Y catalyst of the present invention as a function of TOS at 40 bar (4.0 MPa) and various gas hourly space velocities.

FIG. 8 shows a graphical representation of the methanol yield over Cu/Zn/Zr/Ce/La catalysts of the present invention as a function of TOS at 30 bar (3 MPa), different temperatures, and various gas hourly space velocities.

FIG. 9 shows a graphical representation of the methanol yield over 65% Cu/20% Zn/5% Zr/5% Ce/5% La catalyst of the present invention as a function of TOS at 40 bar (4 MPa) and different GHSV.

FIG. 10 shows a graphical representation of the methanol yield over Cu/Zn/Zr/Ce/Ba catalysts as a function of TOS at 30 bar (3 MPa) and different temperature and GHSV.

FIG. 11 shows a graphical representation of the methanol yield over 60% Cu/20% Zn/10% Zr/5% Ce/5% Ba catalyst of the present invention as a function of TOS at 40 bar (4.0 MPa) and different GHSV.

FIG. 12 shows a graphical representation of the methanol yield and carbon dioxide conversion Cu/Zn/Zr/Ce/Rb catalyst as a function of TOS at 40 bar (4.0 MPa) and different temperatures and GHSV.

FIG. 13 shows a graphical representation of the methanol yield and carbon dioxide conversion Cu/Zn/Zr/Ce/Tb catalyst as a function of TOS at 40 bar (4.0 MPa) and different temperatures and GHSV.

FIG. 14 shows a graphical representation of the methanol yield and carbon dioxide conversion Cu/Zn/Zr/Ce/Sr catalyst as a function of TOS at 40 bar (4.0 MPa) and different GHSVs.

FIG. 15 shows a graphical representation of the methanol yield over 55% Cu/20% Zn/10% Zr/5% Ce/5% Y/5% La catalyst of the present invention as a function of TOS at 40 bar (4.0 MPa), gas hourly space velocity of 5400 h⁻¹, and different temperatures.

### DETAILED DESCRIPTION OF THE INVENTION

A discovery has been made that provides stable, highly active catalysts for the direct catalytic conversion of carbon dioxide to methanol in one pass via a hydrogenation reaction. The invention provides an elegant way to provide a cost-effective method to convert carbon dioxide (CO₂) to methanol and to reduce greenhouse gas emissions while at the same time use a waste product (e.g., CO₂) as an inexpensive and readily available feedstock. The methanol produced from this process can be free, or substantially free, of by-products other than carbon monoxide and water. The discovery is premised on the use of a catalyst that is resistant to water, while catalyzing the reverse water-gas shift reaction (See, reaction scheme (2)) and the production of methanol from carbon dioxide at higher yield at low temperatures. Thus, multi-step reactors are not necessary, thereby increasing the efficiency of the process. By way of example, the current invention provides a substantial improvement over current syngas technologies by providing a process to manufacture methanol without the use of expensive and inefficient equipment (e.g. a reformer that costs approximately 60% of the total methanol plant in current commercial processes). The current embodiments also provide a process to manufacture methanol with higher purity that reduces purification time and cost in comparison to that from the currently available routes. The catalysts of the present invention can be multicomponent heterogeneous mixed metal oxides prepared by gel oxalate co-precipitation. In particular, the discovery is premised on the use of metal oxides that promote storing and releasing of oxygen (e.g., Zn, Zr, Ce, or combinations thereof) in the catalyst in combination with other catalytic metals (e.g., Cu, Y, Ba, Rb, Tb, Sr, Mo, or combinations thereof). Specifically, the presence of the oxygen storage metal oxides in the current catalysts can promote the removal of water by CO in the water-gas shift reaction, stabilize the dispersion of copper, and store and release oxygen under oxidizing and reducing conditions. Thus, these metal oxides, preferably cerium oxide, can be employed in the current embodiments to help protect and stabilize the catalyst active sites for CO₂ hydrogenation to methanol. The catalysts of this invention can also be stable for extended periods during use (e.g., time on stream).

These and other non-limiting aspects of the present invention are discussed in further detail in the following sections.

### A. Mixed Metal Oxide Catalyst

The catalysts of the present invention are capable of producing methanol from carbon dioxide and hydrogen in a single pass. One or more of these catalysts can include a heterogeneous mixed metal oxide catalyst that can contain metals (e.g., metals in reduced form), metal compounds (e.g., metal oxides) or mixtures thereof (“collectively metals”) of Column 1 or 2 metals, transition metals, and lanthanides (atomic number 57-71) of the Periodic Table. The metals in the catalyst can exist in one or more oxidation states. A non-limiting example of a Column 1 and 2 metals includes rubidium (Rb), barium (Ba) and strontium (Sr). Non-limiting examples of transition metals include ytterbium (Y), titanium (Ti), zirconium (Zr), molybdenum (Mo), tungsten (W), copper (Cu), silver (Ag), and zinc (Zn). Non-limiting examples of the lower lanthanides include lanthanum (La), cerium (Ce) and terbium (Tb). Preferably, the mixed metal oxide catalyst includes copper, zinc, zirconium, cerium, and M, where M is yttrium, barium, rubidium, terbium, strontium, or molybdenum. In one particular instance, the mixed metal oxide catalyst includes copper, zinc, zirconium, cerium, and yttrium. The mixed metal oxide catalyst can include copper (Cu), M¹ oxides, and M² oxides, where M¹ can be Zn, Zr, Ce, or any combination thereof, and M² is Y, Ba, Rb, Tb, Sr, or Mo, or any combination thereof, with the proviso that M² is not Y when the mixed metal oxide catalyst is [Cu/Zn/M²]Oₙ or [Cu/Zr/M²]Oₙ, where n is determined by the oxidation states of the other elements. The catalyst can be [Cu/Zn/Zr/Ce/M²]Oₙ mixed metal oxide having a general formula of: [CuₐZn_{b}Zr_{c}Ce_{d}Mₑ²]Oₙ where a is 25 to 80, 30 to 70, or 40 to 60, or any range or number there between (e.g., 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80), b is 1 to 57, 5 to 50, 10 to 40, 15 to 30, or any range or number there between (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57), c is 1 to 30, 5 to 25, or 10 to 20 or any number there between (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30), d is 1 to 30, (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30), and e is 1 to 40, 5 to 30, 10 to 20 or any number there between (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40). M² of the mixed metal oxide catalyst can be Y, Ba, Rb, Tb, Sr, Mo or a mixture thereof. In some aspects the catalyst is a [Cu/Zn/M²]Oₙ mixed metal oxide having a general formula of [CuₐZn_{b}M_{c}²]Oₙ, M² is Ba, Rb, Tb, Sr, or Mo, or any combination thereof, where a is 25 to 80, b is 1 to 57, and c is 1 to 30. In another aspect of the invention, the mixed metal catalyst can be a [CuₐZr_{b}M_{c}²]Oₙ metal oxide having the general formula of [CuₐZr_{b}M_{c}²]Oₙ where a is 25 to 80, b is 1 to 57, and c is 1 to 30. The catalyst can have an atomic ratio of metals ranging from about 1 to about 90. For example, in one aspect, the atomic ratio of a Cu/Zn/Zr/Ce/Y catalyst can range from about 5-80:5-30:5-25:1-20:1-20, or 25-80:1-57:1-30:1-30:1-40, preferably about 55:10:15:10:10, about 60:20:10:5:5, about 60:15:10:5:10, about 60:15:10:10:5, and about 65:20:5:5:5. In another aspect, the atomic ratio of a Cu/Zn/Zr/Ce/La catalyst ranges from about 10-80:5-30:1-20:1-20:1-20, or 25-80:1-57:1-30:1-30:1-40, preferably about 45:20:15:10:10, about 60:10:10:10:10, and about 65:20:5:5:5. In another aspect, the atomic ratio of a Cu/Zn/Zr/Ce/Y/La catalyst ranges about 5-80:5-30:5-20:1-15:1-15:1-15, or 25-80:1-57:1-30:1-30:1-40, preferably about 55:20:10:5:5:5. In yet another aspect, the atomic ratio of a Cu/Zn/Zr/Ba catalyst ranges from about 10-80:5-30:5-20:1-20:1-20, or 25-80:1-57:1-30:1-30:1-40, preferably about 55:10:15:10:10, about 60:15:10:10:5, and about 60:20:10:5:5. In still another aspect, the atomic ratio of a Zn/Zr/Ce/Rb ranges from about 10-80:5-30:5-20:1-20:1-20, or 25-80:1-57:1-30:1-30:1-40, preferably about 45:20:15:10:10. In some embodiments, the catalyst is Cu/Zn/Zr/Ce/Sr, preferably 45 at. % Cu/20 at. % Zn/15 at. % Zr/10 at. % Ce/10 at. % Sr.

Copper loading in the catalyst can be from 1 mole % to about 60 mole %, from about 20 mole % to about 60 mole %, and preferably from about 40 mole % to about 60 mole %. The metals used to prepare the catalyst of the present invention can be provided in various oxidation states such as metallic, oxide, hydrate, or salt forms typically depending on the propensity of each metals stability and/or physical/chemical properties. Preferably, the metals or metal oxides used in the preparation of the mixed metal oxide catalyst can be provided in stable oxidation states as complexes with monodentate, bidentate, tridentate, or tetradendate coordinating ligands. Non-limiting examples of ligands include such as for example iodide, bromide, sulfide, thiocyanate, chloride, nitrate, azide, fluoride, hydroxide, oxalate, water, isothiocyanate, acetonitrile, pyridine, ammonia, ethylenediamine, 2,2′-bipyridine, 1,10-phenanthroline, nitrite, triphenylphosphine, cyanide, or carbon monoxide. In a preferred aspect, the mixed metal oxides used to prepare the catalysts of the current invention can be provided as nitrate, nitrate hydrates, nitrate trihydrates, and nitrate hexahydrates. By way of example, copper (II) nitrate trihydrate, zinc nitrate hexahydrate, zirconium (IV) oxynitrate hydrate, cerium (III) nitrate hexahydrate, yttrium (III) nitrate hexahydrate, lanthanum (III) nitrate hexahydrate, and barium nitrate can be used. Non-limiting examples of commercial sources of the above-mentioned metals and metal oxides, and metal complexes are Sigma Aldrich® (U.S.A), Acros Organics (Thermo Fisher Scientific, U.S.A.), and Alfa Aesar (U.S.A.).

### B. Methods of Making Mixed Metal Oxide Catalysts

The catalyst can be prepared by co-precipitation of metals, or salts thereof, from a solution containing oxalic acid followed by calcination of the precipitate. Co-precipitation is the simultaneous precipitation of one or more metal salts from a solution to form a mixed metal catalyst precursor. By way of example, a catalyst of the present invention can be prepared by gel oxalate co-precipitation. In a gel oxalate co-precipitation method, a solution of the desired metal salt or mixture of metal precursor material (e.g., metal nitrate salts of Cu, Zn, Zr, Ce, with Ba, La, Y, Sr, Tb, Rb, or any combination thereof) can be obtained by mixing the metal salt precursor material in the appropriate molar ratios together in a solvent (e.g., water or alcohol). A second mixture that includes oxalic acid dissolved in a solvent (e.g., methanol, ethanol, butanol, etc.) can be prepared. The two solutions can be added together slowly at room temperature (e.g., 20° C. to 35° C.) under agitation, preferably vigorous agitation. The contact the oxalic acid solution with the metal salt solution promotes precipitation of the catalyst precursor having (e.g., mixed metal oxalates). The formed precipitate can be collected by standard techniques, such as decanting, filtration, or centrifuging. In a preferred aspect, the precipitate can be centrifuged at a range from about 3000 rpm to about 7000 rpm, from about 4000 rpm to about 6000 rpm, and preferably about 5000 rpm for anywhere between 10 minutes and 30 minutes, preferably 15 minutes. The separated precipitate can be dried to remove water and/or solvent. By way of example, the precipitate can be dried overnight at temperature from about 100° C. to about 120° C., preferably 110° C. overnight to obtain a dried catalyst precursor. The catalyst precursor can be calcined (e.g., heated in the presence of an oxidant) to obtain the mixed metal oxide catalyst of the present invention. By way of example, the catalyst precursor can be heated for 2 to 6 hours at a temperature of 250 to 450° C. under a flow of air to obtain a mixed metal oxide catalyst. The mixed metal oxide catalyst can be reduced (e.g., subjected to a hydrogen flow for about 2 to 3 hours at a temperature of 180° C. to 350° C.).

In some aspects, the catalysts of the present invention are prepared under oxidative conditions (e.g., calcination) and the metals included in the heterogeneous catalyst are present in higher oxidation states, for example as oxides. Prior to being used as hydrogenation catalysts for the direct conversion of CO₂ to methanol, the catalyst can be treated under reducing conditions to convert some or all of the metals to a lower, more active, oxidation state (e.g. a zero valence). In a preferred aspect, the prepared mixed metal oxide catalysts of the current invention are subjected to reducing conditions (e.g., a gaseous hydrogen stream) within the reactor or separately at a temperature ranging from about 220° C. to about 300° C., from about 250° C. to about 290° C. and preferably around 270° C. under 10 vol. % to 50 vol. % H₂ in Ar, 20 vol. % to 40 vol. % H₂ in Ar, and preferably 25 vol. % H₂ in Ar for 1 h to 3 h, and preferably 2 h.

The catalysts of the present invention can be ground into a fine powder, micronized or nanonized to desired mesh particle size distributions, or pressed into pellets, crushed, and sieved to particle size ranges from about 100 μm to about 600 μm, from about 200 μm to about 500 μm, and preferably between 250 μm and 425 μm. Without wishing to be bound by theory, it is believed that the catalyst activity depends on the particle size of the metals in the mixed metal oxide catalyst, which depends mainly on electronic effects, as the electron density at the active sites (on the surface) can vary due to particle size. This effect can be closely related to particle shape and the number of low coordination sites (edges and corners) on the surface as well as the composition of the catalyst. The average CuO particle size can range from 10.5 to 11.4 nm. The mixed metal oxide catalyst can include copper in the Cu⁰ and Cu⁺¹ oxidation states. The average Cu⁰ species particle size in the catalyst can range from 10.5 to 12.5 nm, and the average Cu⁺¹ species particle size can range 8 to 10.5 nm.

In other instances, the catalysts of present invention can also be prepared by a solid transformation such as found in the preparation of epitaxial metals, unsupported bulk metals, amorphous alloys, or colloidal metals.

### C. Hydrogen/Carbon Dioxide Stream

Carbon dioxide and hydrogen used in the present invention can be obtained from various sources. In one non-limiting instance, the carbon dioxide can be obtained from a waste or recycle gas stream (e.g., from a plant on the same site, like for example from ammonia synthesis) or after recovering the carbon dioxide from a gas stream. A benefit of recycling such carbon dioxide as a starting material in the process of the invention is that it can reduce the amount of carbon dioxide emitted to the atmosphere (e.g., from a chemical production site). The hydrogen may be from various sources, including streams coming from other chemical processes, like water splitting (e.g., photocatalysis, electrolysis, or the like), syngas production, ethane cracking, methanol synthesis, or conversion of methane to aromatics. Preferably, the hydrogen is obtained by water splitting. The H₂/CO₂ reactant gas stream ratio for the hydrogenation reaction can range from 1 to 5, or 1:1, 2:1, 3:1, 4:1, or 5:1, preferably 3:1 to 5:1 with the remainder of the reactant gas stream comprising another gas or gases provided the gas or gases are inert, such as argon (Ar) or nitrogen (N₂), and do not negatively affect the reaction. All possible percentages of CO₂+H₂+inert gas are anticipated in the current embodiments as having the described H₂/CO₂ ratios herein. For example, in one instance the reactant stream includes 22 vol. % CO₂, 67 vol. % H₂, and 11 vol. % Ar. Preferably, the reactant mixture is highly pure and substantially devoid of water or steam. In some embodiments, the carbon dioxide can be dried prior to use (e.g., pass through a drying media) or contains a minimal amount of or no water.

### D. Methanol Production System

Conditions sufficient for the hydrogenation of CO₂ to methanol include temperature, time, space velocity, and pressure. The temperature range for the hydrogenation reaction can range from about 200° C. to 300° C., from about 210° C. to 280° C., preferably from about 220° C. to about 260° C. and all ranges there between including 221° C., 222° C., 223° C., 224° C., 225° C., 226° C., 227° C., 228° C., 229° C., 230° C., 231° C., 232° C., 233° C., 234° C., 235° C., 236° C., 237° C., 238° C., 239° C., 240° C., 241° C., 242° C., 243° C., 244° C., 245° C., 246° C., 247° C., 248° C., 249° C., 250° C., 251° C., 252° C., 253° C., 254° C., 255° C., 256° C., 257° C., 258° C., and 259° C. The gas hourly space velocity (GHSV) for the hydrogenation reaction can range from about 2,500 h⁻¹ to about 20,000 h⁻¹, from about 3,500 h⁻¹ to about 10,000 h⁻¹, and preferably from about 4,000 h⁻¹ to about 6,000 h⁻¹. The average pressure for the hydrogenation reaction can range from about 1 bar to about 100 bar (0.1 MPa to 10 MPa), from about 0.2 MPa to about 6 MPa, preferably about 3 MPa to about 5 MPa and all pressures there between including 3.1 MPa, 3.2 MPa, 3.3 MPa, 3.4 MPa, 3.5 MPa, 3.6 MPa, 3.7 MPa, 3.8 MPa, 3.9 MPa, 4.0 MPa, 4.1 MPa, 4.2 MPa, 4.3 MPa, 4.4 MPa, 4.5 MPa, 4.6 MPa, 4.7 MPa, 4.8 MPa, and 4.9 MPa, or more. The upper limit on pressure can be determined by the reactor used. The conditions for the hydrogenation of CO₂ to methanol can be varied based on the type of the reactor.

In another aspect, the reaction can be carried out over the catalyst of the current invention having the particular methanol selectivity and conversion for prolonged periods of time without changing or re-supplying new catalyst or preforming catalyst regeneration. This can be due to the stability or slower deactivation of the catalysts of the present invention. In one aspect, the reaction can be performed where the one pass methanol selectivity is at least 10 to 100%, or at least 25%, at least 15 to 22%, or more. In some instances, the methanol selectivity can be 18 to 21% after 100 hours to 800 hours on the stream. In another aspect, the one pass CO₂ conversion is at least 5% or more, or at least 5% to 99%, 10% to 80%, or 20% to 60%. In some embodiments, the CO₂ conversion is at least 5% to 60% after 100 hours to 800 hours on the stream. The catalysts of the present invention can remain 90 to 99% active, preferably 94 to 98% active, after 350 hours or more of time on the stream. The method can further include collecting or storing the produced methanol along with using the produced methanol as a feed source, solvent or a commercial product. Prior to use, the catalyst can be subjected to reducing conditions to convert the copper oxide (Cu⁺²) to Cu⁺¹ and Cu⁰ species. A non-limiting example of reducing conditions includes flowing a gaseous stream that includes hydrogen gas (e.g., a H₂ and Argon gas stream) at a temperature of 250 to 280° C. for a period of time (e.g., 1, 2, or 3 hours).

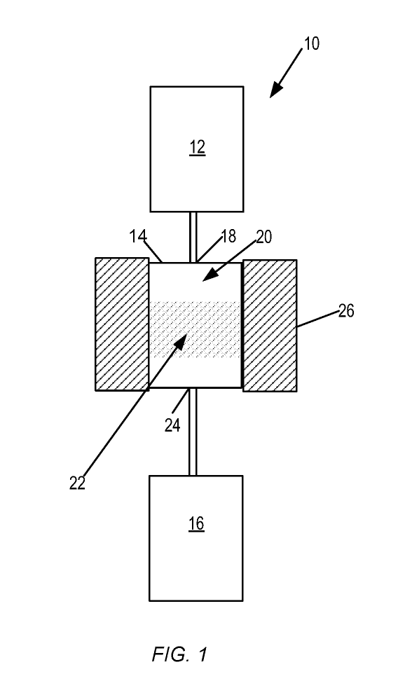

FIG. 1 depicts a system 10 that can be used to convert carbon dioxide (CO₂) and hydrogen (H₂) to methanol using the mixed metal oxide catalysts of the present invention. The system 10 can include an H₂/CO₂ source 12, a reactor 14, and a collection device 16. The H₂/CO₂ source 12 can be configured to be in fluid communication with the reactor 14 via an inlet 18 on the reactor. As explained above, the CO₂/H₂ feed source 12 can be configured such that it regulates the amount of reactant feed entering the reactor 14. As shown, the CO₂/H₂ feed source 12 is one unit feeding into one inlet 18, however, it should be understood that the number of inlets and/or separate feed sources can be adjusted to reactor sizes and/or configurations. The reactor 14 can include a reaction zone 20 having the mixed metal oxide catalyst 22 of the present invention. The reactor can include various automated and/or manual controllers, valves, heat exchangers, gauges, etc. necessary for the operation of the reactor. The reactor can be have the necessary insulation and/or heat exchangers to heat or cool the reactor as necessary. The amounts of the CO₂/H₂ feed and the mixed metal oxide catalyst 22 used can be modified as desired to achieve a given amount of product produced by the system 10. Non-limiting examples of continuous flow reactors that can be used include fixed-bed reactors, fluidized reactors, bubbling bed reactors, slurry reactors, rotating kiln reactors, moving bed reactors or any combinations thereof when two or more reactors are used. In preferred aspects, reactor 14 is a continuous flow fixed-bed reactor. The reactor 14 can include an outlet 24 configured to be in fluid communication with the reaction zone and configured to remove a first product stream comprising methanol from the reaction zone 20. Reaction zone 20 can further include the reactant feed and the first product stream. The products produced can include methanol, carbon monoxide, and water. In some aspects, the catalyst can be included in the product stream. The collection device 16 can be in fluid communication with the reactor 14 via the outlet 24. Both the inlet 18 and the outlet 24 can be opened and closed as desired. The collection device 16 can be configured to store, further process, or transfer desired reaction products (e.g., methanol) for other uses. In a non-limiting example, collection device can be a separation unit or a series of separation units that are capable of separating the liquid components from the gaseous components from the product stream. By way of example, the methanol and water can be condensed from the gas stream. Any unreacted H₂/CO₂ can be recycled and included in the H₂/CO₂ feed to further maximize the overall conversion of H₂/CO₂ to methanol, increases the efficiency and commercial value of the H₂/CO₂ to methanol conversion process of the present invention. The water can be removed from the methanol using known drying/separation methods for the removal of water from methanol. The resulting methanol can be sold, stored or used in other processing units as a feed source. Still further, the system 10 can also include a heating/cooling source 26. The heating/cooling source 26 can be configured to heat or cool the reaction zone 20 to a temperature sufficient (e.g., 220 to 260° C.) to convert the H₂/CO₂ in the H₂/CO₂ feed to methanol. Non-limiting examples of a heating/cooling source 20 can be a temperature controlled furnace or an external, electrical heating block, heating coils, or a heat exchanger.

### EXAMPLES

The present invention will be described in greater detail by way of specific examples. The following examples are offered for illustrative purposes only, and are not intended to limit the invention in any manner. Those of skill in the art will readily recognize a variety of noncritical parameters, which can be changed or modified to yield essentially the same results.

### Catalyst Preparation

### Example 1

### Synthesis of Cu/Zn/Zr/Ce/Y

The catalyst with an atomic ratio of copper, zinc, zirconium, cerium and yttrium of 55:10:15:10:10 were prepared by gel oxalate co-precipitation using 20% excess of oxalic acid. Two separate solutions were prepared (i) a mixture of nitrates (6.27 g of copper (II) nitrate trihydrate, 1.36 g of zinc nitrate hexahydrate and 1.14 g of zirconium (IV) oxynitrate hydrate, 0.93 g of cerium (III) nitrate hexahydrate and 1.29 g of yttrium (III) nitrate hexahydrate) and (ii) oxalic acid (6.12 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 2

### Synthesis of Cu/Zn/Zr/Ce/Y

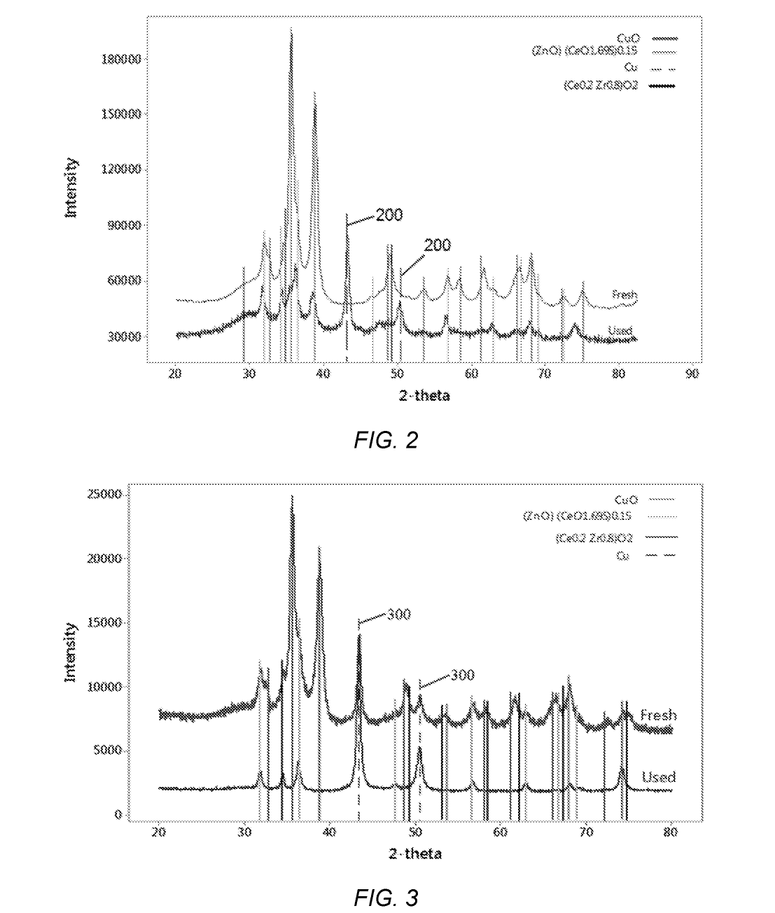

The catalyst with an atomic ratio of copper, zinc, zirconium, cerium and yttrium of 60:20:10:5:5 were prepared by gel oxalate co-precipitation using 20% excess of oxalic acid. Two separate solutions were prepared (i) a mixture of nitrates (6.84 g of copper (II) nitrate trihydrate, 2.73 g of zinc nitrate hexahydrate and 0.76 g of zirconium (IV) oxynitrate hydrate, 0.46 g of cerium (III) nitrate hexahydrate and 0.65 g of yttrium (III) nitrate hexahydrate) and (ii) oxalic acid (6.55 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention. The catalyst particle size was calculated from XRD results by Scherrer formula and was found to be 11.4 nm for Cu²⁺, 10.5 nm for Cu⁺, and 10.5 nm Cu⁰. FIG. 2 is XRD patterns for fresh (top pattern) and used (bottom pattern) for Example 2 catalyst. Lines 200 show peaks specific to metallic copper.

### Example 3

### Synthesis of Cu/Zn/Zr/Ce/Y

The catalyst with an atomic ratio of copper, zinc, zirconium, cerium and yttrium of 60:15:10:5:10 were prepared by gel oxalate co-precipitation using 20% excess of oxalic acid. Two separate solutions were prepared (i) a mixture of nitrates (6.84 g of copper (II) nitrate trihydrate, 2.05 g of zinc nitrate hexahydrate and 0.76 g of zirconium (IV) oxynitrate hydrate, 0.46 g of cerium (III) nitrate hexahydrate and 1.29 g of yttrium (III) nitrate hexahydrate) and (ii) oxalic acid (6.42 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 4

### Synthesis of Cu/Zn/Zr/Ce/Y

The catalyst with an atomic ratio of copper, zinc, zirconium, cerium and yttrium of 60:15:10:10:5 were prepared by gel oxalate co-precipitation using 20% excess of oxalic acid. Two separate solutions were prepared (i) a mixture of nitrates (6.84 g of copper (II) nitrate trihydrate, 2.05 g of zinc nitrate hexahydrate and 0.76 g of zirconium (IV) oxynitrate hydrate, 0.93 g of cerium (III) nitrate hexahydrate and 0.65 g of yttrium (III) nitrate hexahydrate) and (ii) oxalic acid (6.36 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 5

### Synthesis of Cu/Zn/Zr/Ce/Y

The catalyst with an atomic ratio of copper, zinc, zirconium, cerium and yttrium of 65:20:5:5:5 were prepared by gel oxalate co-precipitation using 20% excess of oxalic acid. Two separate solutions were prepared (i) a mixture of nitrates (7.41 g of copper (II) nitrate trihydrate, 2.73 g of zinc nitrate hexahydrate and 0.38 g of zirconium (IV) oxynitrate hydrate, 0.46 g of cerium (III) nitrate hexahydrate and 0.65 g of yttrium (III) nitrate hexahydrate) and (ii) oxalic acid (6.66 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 6

### Synthesis of Cu/Zn/Zr/Ce/La

The catalyst with an atomic ratio of copper, zinc, zirconium, cerium and lanthanum of 45:20:15:10:10 were prepared by gel oxalate co-precipitation using 20% excess of oxalic acid. Two separate solutions were prepared (i) a mixture of nitrates (5.13 g of copper (II) nitrate trihydrate, 2.73 g of zinc nitrate hexahydrate and 1.14 g of zirconium (IV) oxynitrate hydrate, 0.93 g of cerium (III) nitrate hexahydrate and 0.70 g of lanthanum (III) nitrate hexahydrate) and (ii) oxalic acid (5.91 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 7

### Synthesis of Cu/Zn/Zr/Ce/La

The catalyst with an atomic ratio of copper, zinc, zirconium, cerium and lanthanum of 60:10:10:10:10 were prepared by gel oxalate co-precipitation using 20% excess of oxalic acid. Two separate solutions were prepared (i) a mixture of nitrates (6.84 g of copper (II) nitrate trihydrate, 1.36 g of zinc nitrate hexahydrate and 0.76 g of zirconium (IV) oxynitrate hydrate, 0.93 g of cerium (III) nitrate hexahydrate and 0.70 g of lanthanum (III) nitrate hexahydrate) and (ii) oxalic acid (6.04 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 8

### Synthesis of Cu/Zn/Zr/Ce/La

The catalyst with an atomic ratio of copper, zinc, zirconium, cerium and lanthanum of 65:20:5:5:5 were prepared by gel oxalate co-precipitation using 20% excess of oxalic acid. Two separate solutions were prepared (i) a mixture of nitrates (7.41 g of copper (II) nitrate trihydrate, 2.73 g of zinc nitrate hexahydrate and 0.38 g of zirconium (IV) oxynitrate hydrate, 0.46 g of cerium (III) nitrate hexahydrate and 0.35 g of lanthanum (III) nitrate hexahydrate) and (ii) oxalic acid (6.56 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention. The catalyst particle size was calculated from XRD results by Scherrer formula and was found to be 12.5 nm for Cu²⁺, 8 nm for Cu⁺, and 10.5 nm for Cu⁰. FIG. 3 shows XRD patterns for fresh (top pattern) and used (bottom pattern) Example 3 catalyst. Lines 300 show peaks specific to reduced copper.

### Example 9

### Synthesis of Cu/Zn/Zr/Ce/Y/La

The catalyst with an atomic ratio of copper, zinc, zirconium, cerium, yttrium and lanthanum of 55:20:10:5:5:5 were prepared by gel oxalate co-precipitation using 20% excess of oxalic acid. Two separate solutions were prepared (i) a mixture of nitrates (6.27 g of copper (II) nitrate trihydrate, 2.73 g of zinc nitrate hexahydrate and 0.76 g of zirconium (IV) oxynitrate hydrate, 0.46 g of cerium (III) nitrate hexahydrate, 0.65 g of yttrium (III) nitrate hexahydrate and 0.35 g of lanthanum (III) nitrate hexahydrate) and (ii) oxalic acid (6.31 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 10

### Synthesis of Cu/Zn/Zr/Ce/Ba

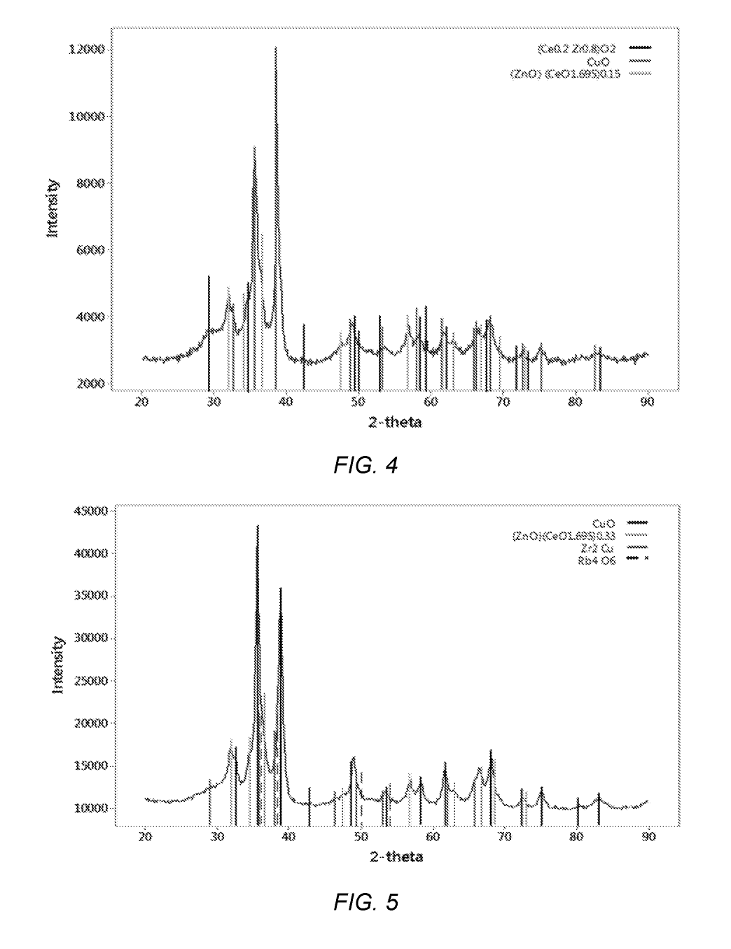

The catalyst with an atomic ratio of copper, zinc, zirconium, cerium and barium of 55:10:15:10:10 were prepared by gel oxalate co-precipitation using 20% excess of oxalic acid. Two separate solutions were prepared (i) a mixture of nitrates (6.27 g of copper (II) nitrate trihydrate, 1.36 g of zinc nitrate hexahydrate and 1.14 g of zirconium (IV) oxynitrate hydrate, 0.93 g of cerium (III) nitrate hexahydrate and 0.57 g of barium nitrate) and (ii) oxalic acid (5.88 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention. FIG. 4 is an XRD pattern of the catalyst.

### Example 11

### Synthesis of Cu/Zn/Zr/Ce/Ba

The catalyst with an atomic ratio of copper, zinc, zirconium, cerium and barium of 60:15:10:10:5 were prepared by gel oxalate co-precipitation using 20% excess of oxalic acid. Two separate solutions were prepared (i) a mixture of nitrates (6.84 g of copper (II) nitrate trihydrate, 2.05 g of zinc nitrate hexahydrate and 0.76 g of zirconium (IV) oxynitrate hydrate, 0.93 g of cerium (III) nitrate hexahydrate and 0.29 g of barium nitrate) and (ii) oxalic acid (6.24 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 12

### Synthesis of Cu/Zn/Zr/Ce/Ba

The catalyst with an atomic ratio of copper, zinc, zirconium, cerium and barium of 60:20:10:5:5 were prepared by gel oxalate co-precipitation using 20% excess of oxalic acid. Two separate solutions were prepared (i) a mixture of nitrates (6.84 g of copper (II) nitrate trihydrate, 2.73 g of zinc nitrate hexahydrate and 0.76 g of zirconium (IV) oxynitrate hydrate, 0.46 g of cerium (III) nitrate hexahydrate and 0.29 g of barium nitrate) and (ii) oxalic acid (6.43 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention. FIG. 5 shows an XRD pattern of the Cu/Zn/Zr/Ce/Ba catalyst.

### Example 13

### Synthesis of Cu/Zn/Zr/Ce/Rb

The catalyst with an atomic ratio of copper, zinc, zirconium, cerium and rubidium of 45:20:15:10:10 were prepared by gel oxalate co-precipitation using 20% excess of oxalic acid. Two separate solutions were prepared (i) a mixture of nitrates (5.13 g of copper (II) nitrate trihydrate, 2.73 g of zinc nitrate hexahydrate and 1.14 g of zirconium (IV) oxynitrate hydrate, 0.93 g of cerium (III) nitrate hexahydrate and 0.52 g of rubidium nitrate) and (ii) oxalic acid (6.20 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 14

### Synthesis of Cu/Zn/Zr/Ce/Tb

The catalyst with an atomic ratio of copper, zinc, zirconium, cerium and terbium of 45:20:15:10:10 were prepared by gel oxalate co-precipitation using 20% excess of oxalic acid. Two separate solutions were prepared (i) a mixture of nitrates (5.13 g of copper (II) nitrate trihydrate, 2.73 g of zinc nitrate hexahydrate and 1.14 g of zirconium (IV) oxynitrate hydrate, 0.93 g of cerium (III) nitrate hexahydrate and 0.86 g of terbium nitrate hexahydrate) and (ii) oxalic acid (5.96 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 15

### Synthesis of Cu/Zn/Zr/Ce/Sr

The catalyst with an atomic ratio of copper, zinc, zirconium, cerium and terbium of 45:20:15:10:10 were prepared by gel oxalate co-precipitation using 20% excess of oxalic acid. Two separate solutions were prepared (i) a mixture of nitrates (5.13 g of copper (II) nitrate trihydrate, 2.73 g of zinc nitrate hexahydrate and 1.14 g of zirconium (IV) oxynitrate hydrate, 0.93 g of cerium (III) nitrate hexahydrate and 0.72 g of strontium nitrate) and (ii) oxalic acid (5.91 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Catalyst Testing

### General Procedure

Catalyst testing was performed in a high throughput reactor system provided by HTE Company, Germany. The reactors are fixed bed type reactor with 0.5 cm inner diameter and 60 cm in length. Gas flow rates were regulated using Brooks SLA5800 mass flow controllers. Reactor pressure was maintained by restricted capillary before and after the reactor. The reactor temperature was maintained by an external, electrical heating block. The effluent of the reactors is connected to Agilent gas chromatography (GC) 7867 A for online gas analysis. Catalysts were pressed into pellets then crushed and sieved between 250-425 μm. A 0.25 ml of catalyst sieved fraction was placed on top of inert material inside the reactor. Prior to the reaction test, the catalyst in oxidized state was reduced at 270° C. under 25 vol. % H₂ in Ar for 2 h. A mixture of 22 vol. % CO₂+67 vol. % H₂+11 vol. % Ar with gas hourly space velocity (GHSV)=2500, 5000, or 10000 h⁻¹ was introduced into the reactor at 30 and 40 bar and different reaction temperature (e.g., 220, 230, and 240° C.). Argon was used as an internal standard for GC analysis. CO₂ conversion as well as methanol selectivity and yield were calculated as follows:

$${X}_{CO 2} = \frac{[ CO ] + [ {CH}_{4} ] + 2 \times [ {C}_{2} {H}_{6} ] + [ {CH}_{3} OH ]}{{[ {CO}_{2} ]}_{ i n}}$$

$${S}_{CH 3 OH} = \frac{[ {CH}_{3} OH ]}{[ CO ] + [ {CH}_{4} ] + 2 \times [ {C}_{2} {H}_{6} ] + [ {CH}_{3} OH ]}$$

$${Y}_{CH 3 OH} = {X}_{CO 2} \times {S}_{CH 3 OH}$$

All catalysts that were tested in this invention showed high catalytic activity and stability. The commercial catalyst showed about 6% reduction in its activity over 350 h while the best performance catalyst of this invention showed only about 2.5% reduction in its activity over same period of time. The commercial catalyst was obtained from Süd-Chemie (Germany) and had the following composition: 60 wt % CuO/30 wt % ZnO/7.5 wt % Al₂O₃.

### Example 16

### Cu/Zn/Zr/Ce/Y Testing

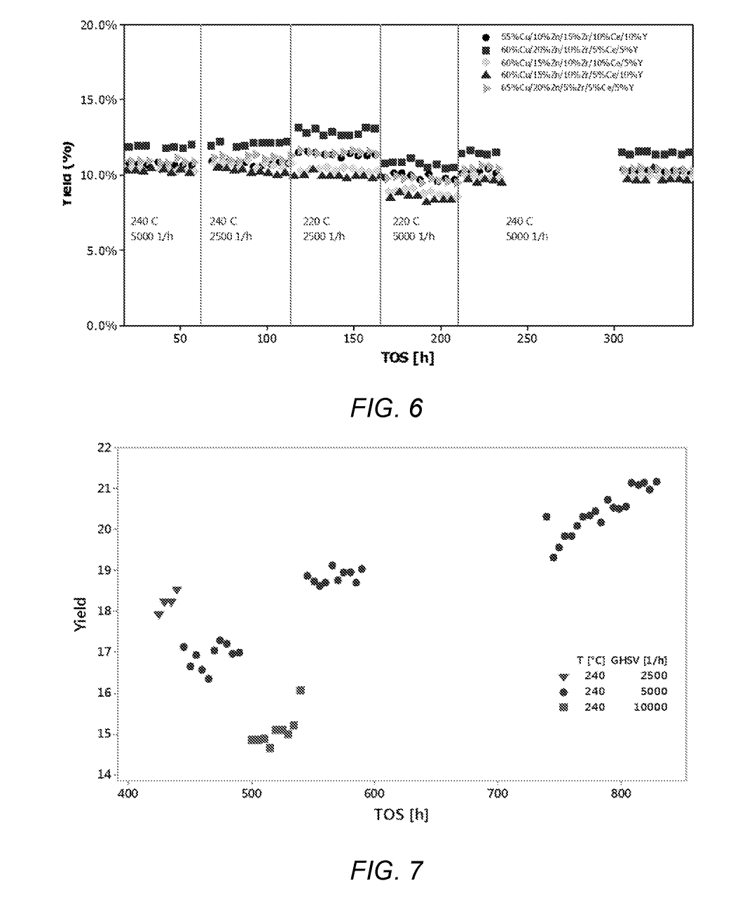

Cu/Zn/Zr/Ce/Y catalysts of the present invention at various atomic ratios (Examples 1-5) were tested under the conditions described in the general procedure. FIG. 6 shows the activity, i.e., methanol yield versus time on stream (TOS), of the catalysts of Examples 1-5 at different temperatures and GHSVs. Circle line monikers is the Example 1 catalyst data, square line monikers is the Example 2 catalyst data, diamond line monikers is the Example 4 catalyst data, triangle line monikers is the Example 3 catalyst data, and side triangle line monikers is Example 5 catalyst data. Table 1 lists the data for FIG. 6. FIG. 7 shows the specific activity of the catalyst prepared in Example 2 (60% Cu/20% Zn/10% Zr/5% Ce/5% Y by weight) at different gas hourly space velocities. Upside down triangle line monikers are data at a GSHV of 2500 1/h, circle line monikers are data at a GSHV of 5000 1/h, and square line monikers are data at a GSHV of 10,000 1/h. The highest methanol yield with this catalyst was shown to be 21% at H₂/CO₂ of 3:1, temperature of 240° C., pressure of 40 bar, and gas hourly space velocity of 5000 h⁻¹.

TABLE 1

| | |
|---|---|
| Catalyst | Methanol Yield (%) |

Reaction Conditions: T = 240° C., P = 40 bar GHSV = 5000 h⁻¹
(atomic %)

| | |
|---|---|
| 55%Cu/10%Zn/15%Zr/10%Ce/10%Y | 10.6 |
| 60%Cu/20%Zn/10%Zr/5%Ce/5%Y | 11.8 |
| 60%Cu/15%Zn/10%Zr/10%Ce/5%Y | 10.3 |
| 60%Cu/15%Zn/10%Zr/5%Ce/10%Y | 10.3 |
| 65%Cu/20%Zn/5%Zr/5%Ce/5%Y | 10.9 |

Reaction Conditions: T = 240° C., P = 40 bar GHSV = 2500 h⁻¹
(mole %)

| | |
|---|---|
| 55%Cu/10%Zn/15%Zr/10%Ce/10%Y | 10.7 |
| 60%Cu/20%Zn/10%Zr/5%Ce/5%Y | 11.9 |
| 60%Cu/15%Zn/10%Zr/10%Ce/5%Y | 10.5 |
| 60%Cu/15%Zn/10%Zr/5%Ce/10%Y | 10.4 |
| 65%Cu/20%Zn/5%Zr/5%Ce/5%Y | 11.3 |

Reaction Conditions: T = 220° C., P = 40 bar GHSV = 2500 h⁻¹
(atomic %)

| | |
|---|---|
| 55%Cu/10%Zn/15%Zr/10%Ce/10%Y | 11.4 |
| 60%Cu/20%Zn/10%Zr/5%Ce/5%Y | 12.8 |
| 60%Cu/15%Zn/10%Zr/10%Ce/5%Y | 10.4 |
| 60%Cu/15%Zn/10%Zr/5%Ce/10%Y | 9.9 |
| 65%Cu/20%Zn/5%Zr/5%Ce/5%Y | 11.5 |

Reaction Conditions: T = 220° C., P = 40 bar GHSV = 5000 h⁻¹
(atomic %)

| | |
|---|---|
| 55%Cu/10%Zn/15%Zr/10%Ce/10%Y | 10 |
| 60%Cu/20%Zn/10%Zr/5%Ce/5%Y | 10.8 |
| 60%Cu/15%Zn/10%Zr/10%Ce/5%Y | 8.8 |
| 60%Cu/15%Zn/10%Zr/5%Ce/10%Y | 8.3 |
| 65%Cu/20%Zn/5%Zr/5%Ce/5%Y | 9.9 |

### Example 17

### Cu/Zn/Zr/Ce/La Testing

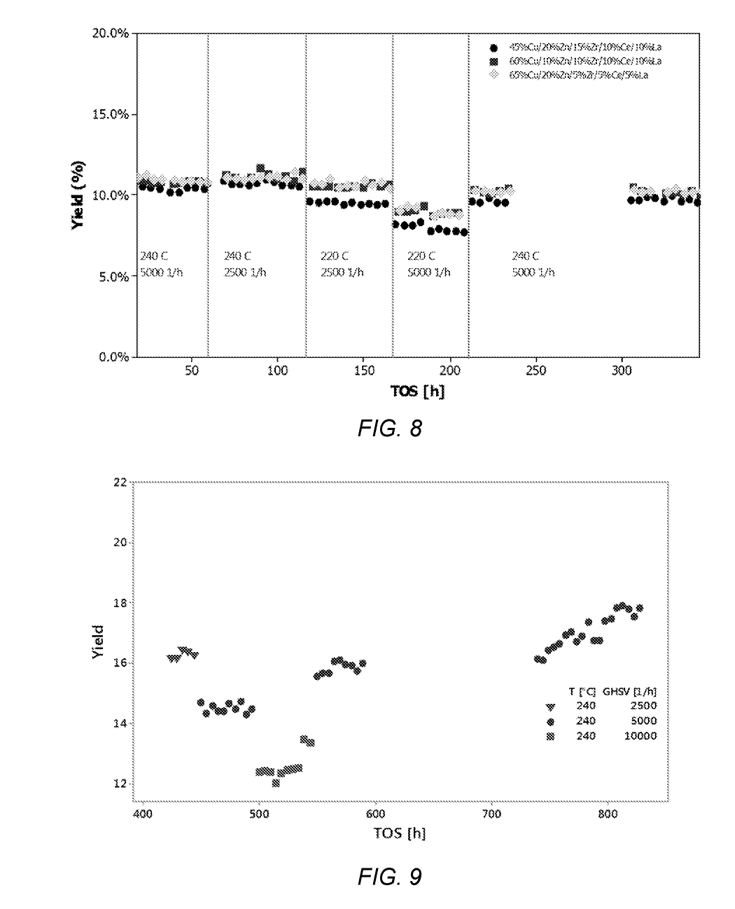

Cu/Zn/Zr/Ce/La catalysts of the present invention at various atomic (molar) loadings (Examples 6-9) were tested using the conditions described in the general procedure. FIG. 8 shows the activity, i.e., methanol yield versus time on stream (TOS), of the catalysts of Examples 6-8 at different temperatures and GHSVs. Circle line monikers is Example 6 catalyst data, square line monikers is Example 7 catalyst data, diamond line monikers is Example 8 catalyst data. Table 2 lists the data for FIG. 8. FIG. 9 shows the specific activity of the Example 8 catalyst (65 at. % Cu/20 at. % Zn/5 at. % Zr/5 at. % Ce/5 at. % La) at different temperatures and GHSVs. Inverted triangle line monikers is data at GHSV of 2500 1/h, circle line monikers is data at a GSHV of 5000 1/h, and square line monikers is data at a GSHV of 10,000 1/h. The highest methanol yield with this catalyst was shown to be 18% at H₂/CO₂ of 3:1, temperature of 240° C., pressure of 40 bar, and gas hourly space velocity of 5000 1/h.

TABLE 2

| | |
|---|---|
| Catalyst | Methanol Yield (%) |

Reaction Conditions: T = 240° C., P = 40 bar GHSV = 5000 h⁻¹
(atomic %)

| | |
|---|---|
| 45%Cu/20%Zn/15%Zr/10%Ce/10%La | 10.4 |
| 60%Cu/10%Zn/10%Zr/10%Ce/10%La | 10.8 |
| 65%Cu/20%Zn/5%Zr/5%Ce/5%La | 11 |

Reaction Conditions: T = 240° C., P = 40 bar GHSV = 2500 h⁻¹
(atomic %)

| | |
|---|---|
| 45%Cu/20%Zn/15%Zr/10%Ce/10%La | 10.7 |
| 60%Cu/10%Zn/10%Zr/10%Ce/10%La | 11.2 |
| 65%Cu/20%Zn/5%Zr/5%Ce/5%La | 11.1 |

Reaction Conditions: T = 220° C., P = 40 bar GHSV = 2500 h⁻¹
(atomic %)

| | |
|---|---|
| 45%Cu/20%Zn/15%Zr/10%Ce/10%La | 9.6 |
| 60%Cu/10%Zn/10%Zr/10%Ce/10%La | 10.5 |
| 65%Cu/20%Zn/5%Zr/5%Ce/5%La | 10.7 |

Reaction Conditions: T = 220° C., P = 40 bar GHSV = 5000 h⁻¹
(atomic %)

| | |
|---|---|
| 45%Cu/20%Zn/15%Zr/10%Ce/10%La | 8.2 |
| 60%Cu/10%Zn/10%Zr/10%Ce/10%La | 9 |
| 65%Cu/20%Zn/5%Zr/5%Ce/5%La | 9.3 |

### Example 18

### Cu/Zn/Zr/Ce/Ba Testing

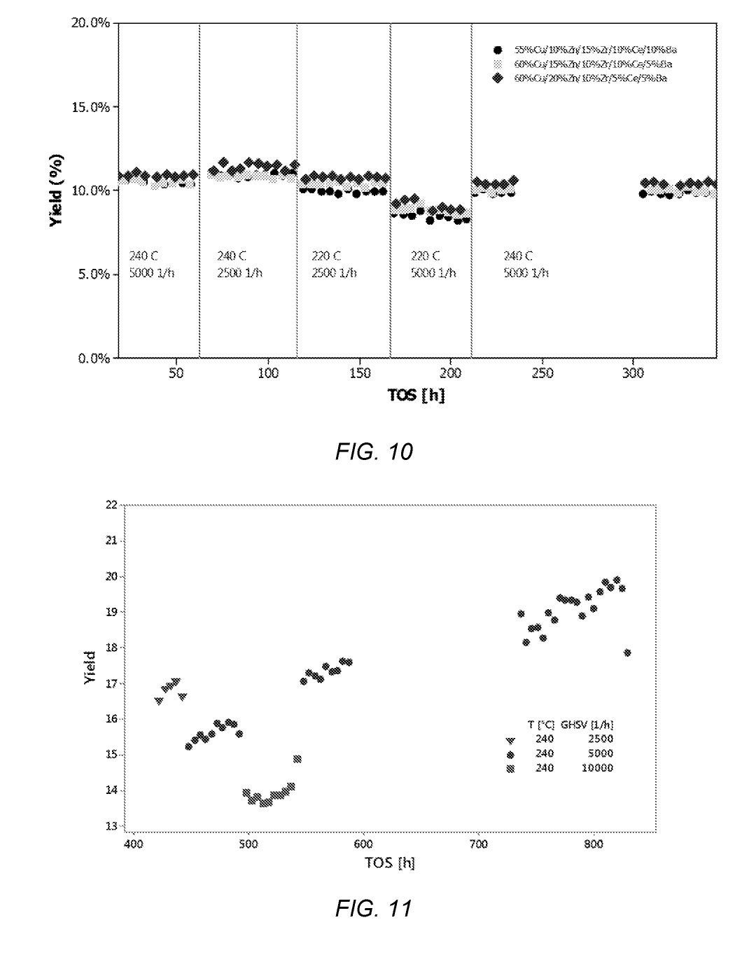

Cu/Zn/Zr/Ce/Ba catalysts of the present invention (Example 12) were tested as described in the general procedure. FIG. 10 shows the activity, i.e., methanol yield versus time on stream (TOS), of the catalysts of Examples 10-12 at different temperatures and gas hourly space velocities. Circle line monikers are Example 10 catalyst data, square line monikers is Example 11 catalyst data, and diamond line monikers is Example 12 catalyst data. Table 3 lists the data for FIG. 10. FIG. 11 shows the specific activity of the Example 12 catalyst (60% Cu/20% Zn/10% Zr/5% Ce/5% Ba) at different temperatures and gas hourly space velocities. Inverted triangle line monikers is data at a GHSV or 2500 1/h, circle line monikers is data at a GHSV of 5000 1/h, and square line monikers is data at a GHSV of 10000 1/h. It was determined that the highest methanol yield with this catalyst was shown to be 20% at H₂/CO₂ of 3, temperature of 240° C., pressure of 40 bar (4.0 MPa), and gas hourly space velocity of 5000 h⁻¹.

TABLE 3

| | |
|---|---|
| Catalyst | Methanol Yield (%) |

Reaction Conditions: T = 240° C., P = 40 bar GHSV = 5000 h⁻¹
(atomic %)

| | |
|---|---|
| 55%Cu/10%Zn/15%Zr/10%Ce/10%Ba | 10.6 |
| 60%Cu/15%Zn/10%Zr/10%Ce/5%Ba | 10.5 |
| 60%Cu/20%Zn/10%Zr/5%Ce/5%Ba | 11 |

Reaction Conditions: T = 240° C., P = 40 bar GHSV = 2500 h⁻¹
(atomic %)

| | |
|---|---|
| 55%Cu/10%Zn/15%Zr/10%Ce/10%Ba | 11 |
| 60%Cu/15%Zn/10%Zr/10%Ce/5%Ba | 10.9 |
| 60%Cu/20%Zn/10%Zr/5%Ce/5%Ba | 11.6 |

Reaction Conditions: T = 220° C., P = 40 bar GHSV = 2500 h⁻¹
(atomic %)

| | |
|---|---|
| 55%Cu/10%Zn/15%Zr/10%Ce/10%Ba | 10 |
| 60%Cu/15%Zn/10%Zr/10%Ce/5%Ba | 10.5 |
| 60%Cu/20%Zn/10%Zr/5%Ce/5%Ba | 10.9 |

Reaction Conditions: T = 220° C., P = 40 bar GHSV = 5000 h⁻¹
(atomic %)

| | |
|---|---|
| 55%Cu/10%Zn/15%Zr/10%Ce/10%Ba | 8.5 |
| 60%Cu/15%Zn/10%Zr/10%Ce/5%Ba | 8.9 |
| 60%Cu/20%Zn/10%Zr/5%Ce/5%Ba | 9 |

### Example 19

### Cu/Zn/Zr/Ce/Rb Testing

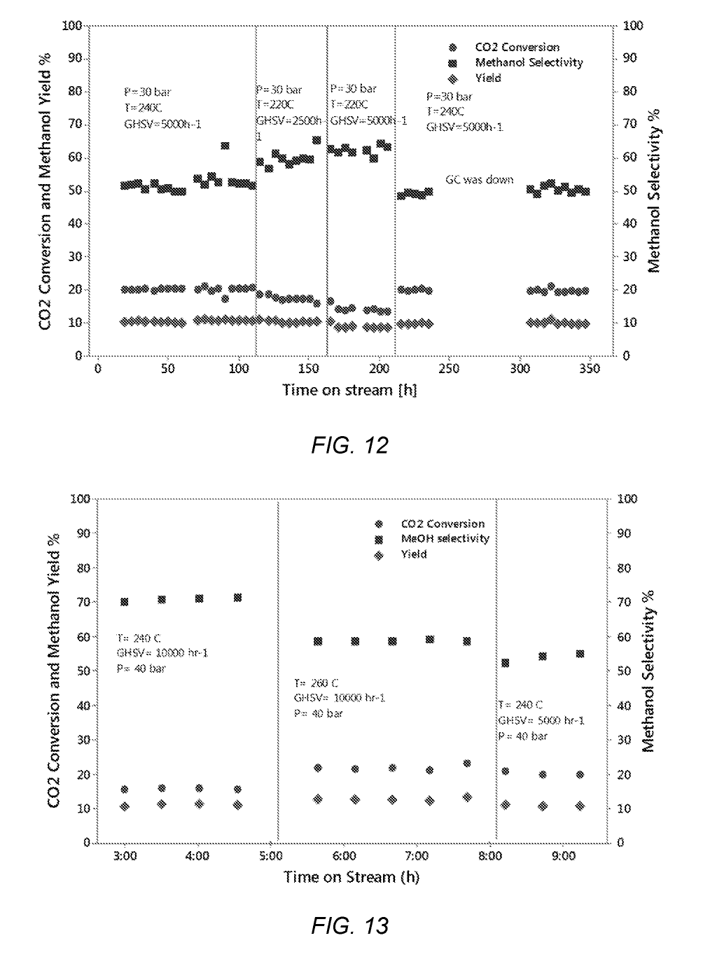

Cu/Zn/Zr/Ce/Rb catalysts of the present invention (Example 14) were tested as described in the general procedure. FIG. 12 shows the activity, i.e., methanol yield versus time on stream (TOS), carbon dioxide conversion and methanol yield for the Example 13 catalyst at different temperatures and gas hourly space velocities. Circle line monikers represent data for carbon dioxide conversion, square line monikers represent data for methanol selectivity, and diamond line monikers represent data for methanol yield. Table 4 lists the data for FIG. 12.

TABLE 4

| | | Methanol |
|---|---|---|
| Catalyst (atomic %) | Reaction conditions | Yield (%) |
| 45%Cu/20%Zn/15% | T = 240 C., P = 30 bar | 10.1 |
| Zr/10%Ce/10%Rb | GHSV = 5000 h⁻¹ | |
| 45%Cu/20%Zn/15% | T = 220 C., P = 30 bar | 10 |
| Zr/10%Ce/10%Rb | GHSV = 2500 h⁻¹ | |
| 45%Cu/20%Zn/15% | T = 220 C., P = 30 bar | 9.8 |
| Zr/10%Ce/10%Rb | GHSV = 5000 h⁻¹ | |

### Example 20

### Cu/Zn/Zr/Ce/Tb Testing

Cu/Zn/Zr/Ce/Tb catalysts of the present invention were tested as described in the general procedure. FIG. 13 shows the activity, i.e., methanol yield versus time on stream (TOS), carbon dioxide conversion and methanol yield for the Example 14 catalyst at different temperatures and gas hourly space velocities. Circle line monikers represent data for carbon dioxide conversion, square line monikers represent data for methanol selectivity, and diamond line monikers represent data for yield. Table 5 lists the data for FIG. 13.

TABLE 5

| | | | | Meth- |
|---|---|---|---|---|
| | | | CO₂ | anol |
| | | | Conver- | Selec- |
| Catalyst (atomic %) | Reaction conditions | Yield | sion | tivity |
| 45%Cu/20%Zn/15% | T = 240 C., P = 40 | 11 | 15.5 | 29 |
| Zr/10%Ce/10%Tb | bar GHSV = | | | |
| | 10000 h⁻¹ | | | |
| 45%Cu/20%Zn/15% | T = 260 C., P = 40 | 12.8 | 21.7 | 58.7 |
| Zr/10%Ce/10%Tb | bar GHSV = | | | |
| | 10000 h⁻¹ | | | |
| 45%Cu/20%Zn/15% | T = 240 C., P = 40 | 11 | 21 | 55 |
| Zr/10%Ce/10%Tb | bar GHSV = | | | |
| | 5000 h⁻¹ | | | |

### Example 21

### Cu/Zn/Zr/Ce/Sr Testing

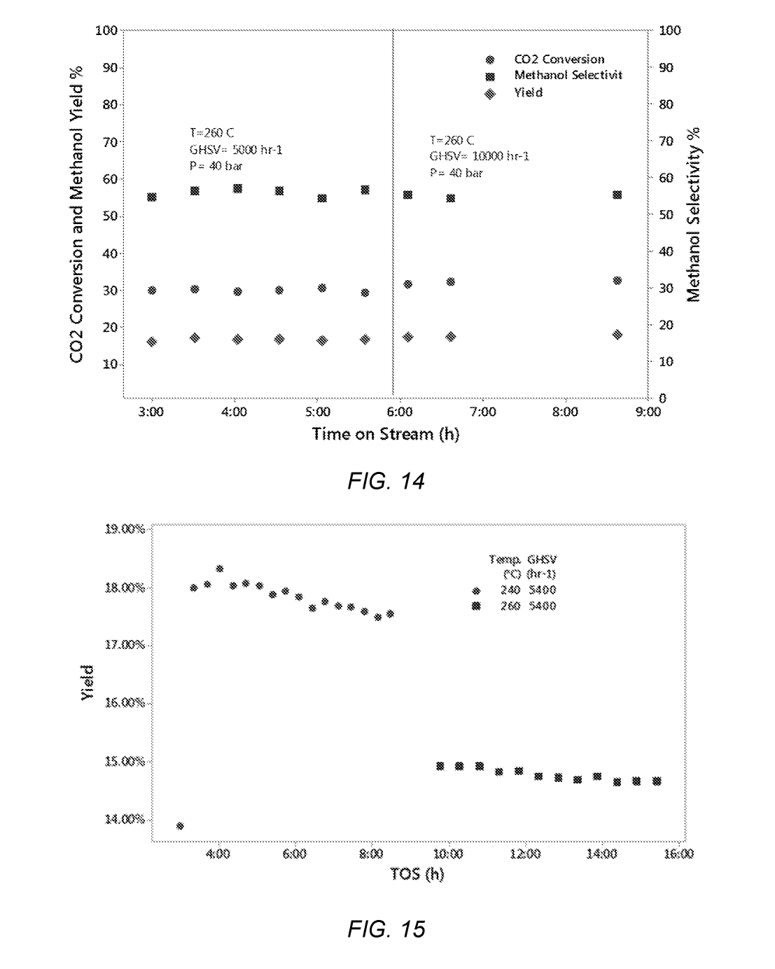

Cu/Zn/Zr/Ce/Sr catalysts of the present invention (Example 15) were tested as described in the general procedure. FIG. 14 shows the activity, i.e., methanol yield versus time on stream (TOS), carbon dioxide conversion and methanol yield for the Example 15 catalyst at different temperatures and gas hourly space velocities. Circle line monikers represent data for carbon dioxide conversion, square line monikers represent data for methanol selectivity, and diamond line monikers represent data for yield.

From the testing of the catalysts, all catalysts of the present invention were able to produce methanol from carbon dioxide at low temperatures and pressures. It was surprisingly found that the catalysts of the present invention that contained yttrium showed higher methanol yield as compare to other catalysts of the present invention. The methanol yield increased from about 13% at 30 bar (3.0 MPa) to about 21% at 40 bar (4.0 MPa). Table 6 lists the data for FIG. 14.

TABLE 6

| | | | | Meth- |
|---|---|---|---|---|
| | | | CO₂ | anol |
| | | | Conver- | Selec- |
| Catalyst (atomic %) | Reaction conditions | Yield | sion | tivity |
| 45%Cu/20%Zn/15% | T = 260 C., P = 40 | 16.8 | 30 | 56 |
| Zr/10%Ce/10%Sr | bar GHSV = | | | |
| | 5000 h⁻¹ | | | |
| 45%Cu/20%Zn/15% | T = 260 C., P = 40 | 17.7 | 32 | 55 |
| Zr/10%Ce/10%Sr | bar GHSV = | | | |
| | 10000 h⁻¹ | | | |

### Example 22

### Cu/Zn/Zr/Ce/Y/La Testing

The Cu/Zn/Zr/Y/La catalysts of Example 9 were tested as described in the general procedure at 240° C. and 260° C. FIG. 15 shows the activity, i.e., methanol yield versus time on stream (TOS), methanol yield for the Example 9 catalyst at different temperatures and constant pressure and gas hourly space velocity. Circle line monikers represent data for methanol yield at 240° C., and square line monikers represent data for methanol yield at 260° C. Table 7 lists the methanol selectivity and the carbon dioxide conversion data for the Example 9 catalyst at a pressure of 40 bar (4.0 MPa), GHSV of 5000 h⁻¹ and different temperatures.

TABLE 7

| | | | | Meth- |
|---|---|---|---|---|
| | | | CO₂ | anol |
| | | | Conver- | Selec- |
| Catalyst (atomic %) | Reaction conditions | Yield | sion | tivity |
| 55%Cu/20%Zn/10% | T = 240 C., P = 40 | 17.9 | 20 | 85 |
| Zr/5%Ce/5%Y/5%La | bar GHSV = | | | |
| | 5000 h⁻¹ | | | |
| 55%Cu/20%Zn/10% | T = 260 C., P = 40 | 14.8 | 18.8 | 79 |
| Zr/5%Ce/5%Y/5%La | bar GHSV = | | | |
| | 5000 h⁻¹ | | | |

1-20. (canceled)

21. A mixed metal oxide catalyst capable of producing methanol (CH₃OH) from carbon dioxide (CO₂) and hydrogen (H₂), the mixed metal oxide catalyst comprising
[CuₐZn_{b}Zr_{c}Ce_{d}Me²]Oₙ
where a is 25 to 80, b is 1 to 57, c is 1 to 30, d is 1 to 30, and e is 1 to 40, and thereof, and
M² is yttrium (Y), barium (Ba), rubidium (Rb), terbium (Tb), strontium (Sr), or molybdenum (Mo), or any combination thereof.

22. The mixed metal oxide catalyst of claim 21, wherein M² is Ba.

23. The mixed metal oxide catalyst of claim 21, wherein M² is Rb.

24. The mixed metal oxide catalyst of claim 21, wherein M² is Tb.

25. The mixed metal oxide catalyst of claim 21, wherein M² is Sr.

26. The mixed metal oxide catalyst of claim 21, wherein M² is Mo.

27. The mixed metal oxide catalyst of claim 21, wherein the catalyst has been calcined for 2 to 6 hours at a temperature of 250 to 650° C.

28. The mixed metal oxide catalyst of claim 27, wherein the catalyst has been reduced for 2 to 3 hours at a temperature of 180 to 350° C.

29. The mixed metal oxide catalyst of claim 21, wherein the catalyst is capable of producing CH₃OH from CO₂ and H₂ in a single pass.

30. A method of producing methanol (CH₃OH) from carbon dioxide (CO₂) and hydrogen (H₂), the method comprising contacting a reactant gas stream that includes CO₂ and H₂ with a mixed metal oxide catalyst of claim 21 under conditions sufficient to produce a product gas stream comprising CH₃OH from hydrogenation of the CO₂.

31. The method of claim 30, wherein CH₃OH is produced from the CO₂ and H₂ in a single pass.

32. The method of claim 32, wherein the reactant gas stream has a ratio of H₂/CO₂ of 1 to 5, preferably 3 to 5 and/or the reaction conditions include a temperature of 200° C. to 300° C., preferably, 220° C. to 260° C., a pressure of 1 bar to 100 bar, preferably 30 bar to 50 bar, and a gas hourly space velocity of 2,500 to 20,000 h⁻¹, preferably of 4,000 V to 6,000 V.

33. The mixed metal oxide catalyst of claim 21, wherein the average CuO particle size is from 10.5 nm to 11.4 nm.

34. The mixed metal oxide catalyst of claim 21, wherein the catalyst is [CuₐZn_{b}Zr_{c}Ce_{d}Yₑ]Oₙ-where a is 25 to 80, b is 1 to 57, c is 1 to 30, d is 1 to 30, and e is 1 to 40.
